(19) United States
(12) Patent Application Publication
(10) Pub. No.: US 2006/0275813 A1
(43) Pub. Date: Dec. 7, 2006

(54) Cartridge for chemical reaction

(75) Inventors: Takeo Tanaami, Tokyo (JP); Hisao Katakura, Tokyo (JP); Saya Satou, Tokyo (JP)

Correspondence Address: SUGHRUE MION, PLLC, 2100 PENNSYLVANIA AVENUE, N.W., SUITE 800, WASHINGTON, DC, 20037, US

(73) Assignee: YOKOGAWA ELECTRIC CORPORATION

(21) Appl. No.: 11/446,225

(22) Filed: Jun. 5, 2006

(30) Foreign Application Priority Data
Jun. 3, 2005 (JP) 2005-163824

(51) Int. Cl. C12Q 1/68; C12M 1/34

(52) U.S. Cl. 435/6; 435/287.2

(57) ABSTRACT

The cartridge for chemical reaction according to the invention is a cartridge for chemical reaction that causes chemical reaction by feeding an internal liquid by way of deformation caused by external forces. The entirety of a target molecule detecting chip is housed in the cartridge for chemical reaction. According to the cartridge for chemical reaction, the entire target molecule detecting chip is housed in the cartridge for chemical reaction so that it supports various forms of target molecule detecting chips. The target molecule detecting chip may be a penetration type chip that allows a solution including target molecules to penetrate the target molecule detecting chip. In this case, the solution may pass inside the penetration type chip by way of deformation of the cartridge for chemical reaction caused by external forces applied to the cartridge for chemical reaction.

### BACKGROUND OF THE INVENTION

The present invention relates to a cartridge for chemical reaction that causes chemical reaction by feeding an internal liquid by way of deformation caused by external forces.

A cartridge has been proposed that supplies an internal liquid by way of deformation caused by external forces with a biopolymer microarray of a slide glass type inserted (refer to Patent Reference 1).

[Patent Reference 1] JP-A-2004-226068

In recent years, a target molecule detecting chip has been developed where detection parts of a large number of different target molecules are densely integrated. Such a molecule detecting chip is formed in an extremely compact form when considering the number of detectable target molecules. Thus, a compact cartridge for chemical reaction is in need that supports such a target molecule detecting chip.

As one type of target molecule detecting chip, a penetration type chip has been developed having a structure that allows a solution to penetrate the chip in its thickness direction. The problem is that the related art cartridge does not support such a penetration type chip. A system has been developed that uses apparatus employing a pump to feed a solution to a penetration type chip to perform hybridization although the result is large-sized apparatus and an expensive system.

### SUMMARY OF THE INVENTION

An object of the invention is to provide a cartridge for chemical reaction that supports detection of a target molecule by using a target molecule detecting chip and that has a compact size.

The present invention provides a cartridge for chemical reaction that causes chemical reaction by feeding an internal liquid by way of deformation caused by external forces, including: a target molecule detecting chip which is entirely housed in the cartridge for chemical reaction, and channels for feeding the internal liquid, being connected to the target molecule detecting chip.

According to the cartridge for chemical reaction of the present invention, the target molecule detecting chip is entirely housed in the cartridge for chemical reaction so that the cartridge for chemical reaction supports various forms of target molecule detecting chips.

The target molecule detecting chip may be a penetration type chip that allows a solution including a target molecule to penetrate the chip.

The target molecule detecting chip may allow the solution to pass through the inside of the penetration type chip by way of deformation caused by external forces applied to the cartridge for chemical reaction.

The present invention provides a cartridge for chemical reaction that causes chemical reaction by feeding an internal liquid by way of deformation caused by external forces, the cartridge for chemical reaction including: a penetration type target molecule detecting chip that allows a solution including a target molecule to penetrate the chip, and channels for feeding the internal liquid, being connected to the target molecule detecting chip, wherein the target molecule detecting chip allows the solution to pass through the inside of the target molecule detecting chip by way of deformation caused by external forces applied to the cartridge for chemical reaction.

According to the cartridge for chemical reaction of the present invention, deformation of the cartridge for chemical reaction caused by external forces applied to the cartridge for chemical reaction allows a solution to pass inside a penetration type target molecule detecting chip. It is thus possible to detect a target molecule with a penetration type target molecule detecting chip by applying external forces.

The target molecule detecting chip may detect any one of DNA, RNA, protein and metabolite as a target molecule.

The cartridge for chemical reaction may include electrodes for applying an electric field to charged molecules inside the cartridge for chemical reaction.

The cartridge for chemical reaction according to the invention houses the entire target molecule detecting chip inside the cartridge for chemical reaction so that it supports various forms of target molecule detecting chips.

Deformation of the inventive cartridge for chemical reaction caused by external forces applied to the cartridge for chemical reaction allows a solution to pass inside a penetration type target molecule detecting chip. It is thus possible to detect a target molecule with a penetration type target molecule detecting chip by applying external forces.

### BRIEF DESCRIPTION OF THE DRAWINGS

FIG. 1 is a plan view of the structure of the cartridge for chemical reaction according to Embodiment 1.

FIG. 2 is a cross sectional view of FIG. 1 taken along line II-II.

FIG. 3A is a perspective view of the structure of a target molecule detecting chip.

FIG. 3B is a cross sectional view of FIG. 3A taken along line B-B.

FIG. 4 shows the operation for solution transfer.

FIG. 5 shows an example where a pretreatment liquid storage part for previously storing a pretreatment liquid and a cleaning solution storage part for previously storing a cleaning liquid.

FIG. 6 shows an example where a target molecule detecting chip is fitted to a component member of the cartridge.

FIG. 7 shows an example where electrodes for accelerating hybridization are formed on the cartridge.

FIG. 8 shows an example where the chip is used as an electrode.

FIG. 9A is a plan view of the structure of the cartridge for chemical reaction according to Embodiment 2.

FIG. 9B is a cross sectional view of FIG. 9A taken along line IXb-IXb.

### DETAILED DESCRIPTION OF THE PREFERRED EMBODIMENTS

Embodiments of the cartridge for chemical reaction according to the invention will be described.

### Embodiment 1

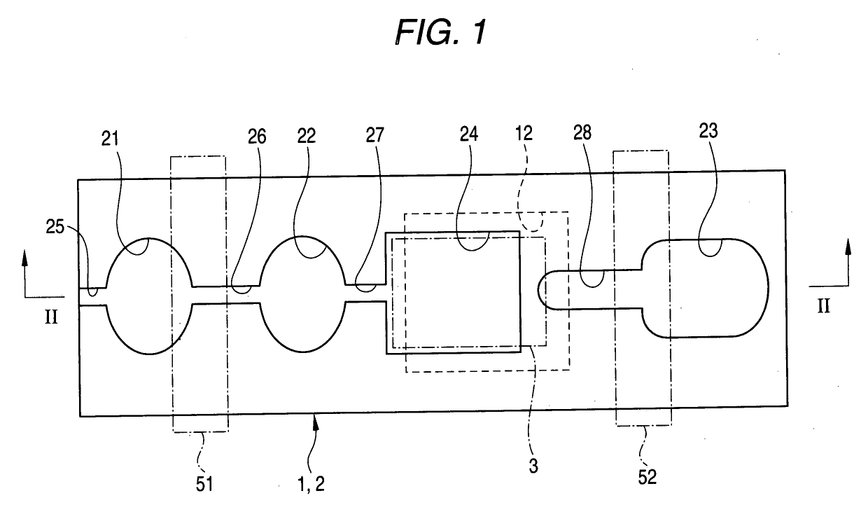

Embodiment 1 of the cartridge for chemical reaction according to the invention will be described referring to FIGS. 1 through 8.

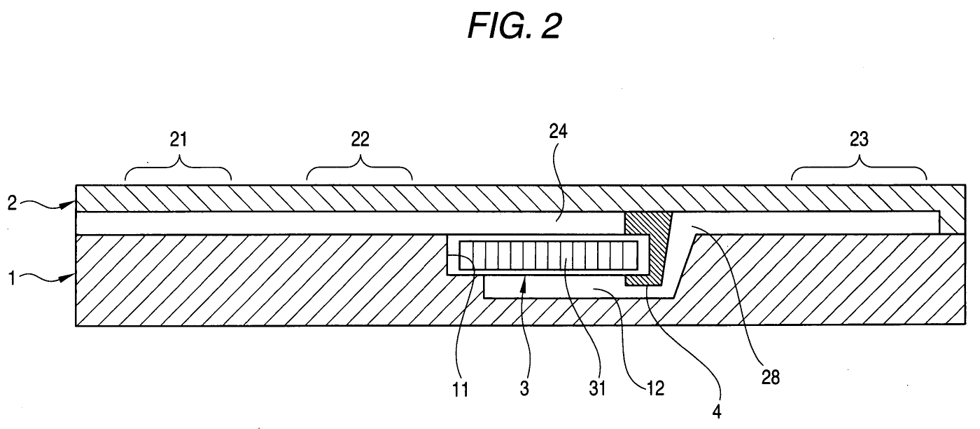

FIG. 1 is a plan view of the structure of the cartridge for chemical reaction according to this embodiment. FIG. 2 is a cross sectional view of FIG. 1 taken along line II-II.

As shown in FIGS. 1 and 2, the cartridge for chemical reaction according to this embodiment comprises a substrate 1 and an elastic member 2 overlaid on the substrate 1.

On the rear surface (bottom surface in FIG. 2) of the elastic member 2 is formed a concave part of a predetermined shape recessed toward the front surface (top surface in FIG. 2). This concave part produces space between the cartridge substrate 1 and the elastic member 2 to constitute chambers 21, 22, 23 for storing a solution, a supply part 24 for supplying a solution to a target molecule detecting chip 3 described later, and channels 25, 26, 27, 28 as shown in FIGS. 1 and 2. As shown in FIG. 1, the channel 25, the chamber 21, the channel 26, the chamber 22, the channel 27 and the supply part 24 are serially interconnected.

In the areas other than the concave part, the cartridge substrate 1 and the elastic member 2 are bonded together. This encloses a solution stored in the concave part inside the cartridge thus preventing leakage to the outside.

As shown in FIGS. 1 and 2, on the substrate 1 are formed a storage part 11 storing the target molecule detecting chip 3 and a discharge part 12 for storing a solution discharged from the target molecule detecting chip 3, the storage part 11 provided on the rear surface of the target molecule detecting chip 3 (on the bottom surface in FIG. 2). On the rear surface of an elastic member 2 is fixed a support member 4 for supporting the target molecule detecting chip 3. The target molecule detecting chip 3 stored in the storage part 11 is bonded to the cartridge substrate 1 and the support member 4 and fixed inside the cartridge.

With this structure, the target molecule detecting chip 3 is fixed inside the cartridge while preventing leakage of solution in the direction of thickness of the target molecule detecting chip 3 from around the target molecule detecting chip 3. As shown in FIG. 2, the discharge part 12 of the cartridge substrate 1 is in communication with the channel 28.

The target molecule detecting chip 3 is a penetration type chip that detects a target molecule by way of hybridization.

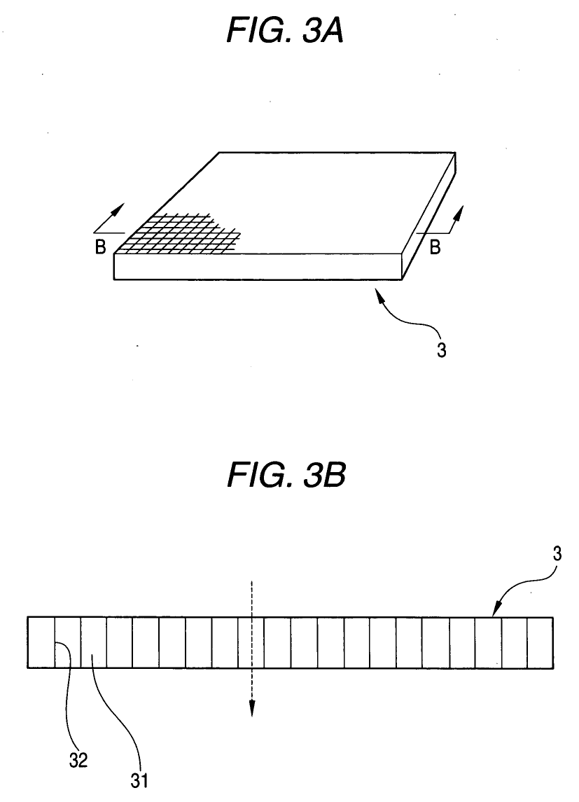

FIG. 3 shows the structure of the target molecule detecting chip 3. FIG. 3A is a perspective view and FIG. 3B is a cross sectional view of FIG. 3A taken along line B-B. As shown in FIGS. 3A and 3B, in the target molecule detecting chip 3, detection parts 31 to which probes corresponding to individual target molecules are fixed are two-dimensionally arranged and the detection parts 31 are segmented by partition walls 32. The detection parts 31 detect target molecules in a solution that penetrates the target molecule detecting chip 3 in its thickness direction (vertical direction in FIG. 3B) by way of hybridization.

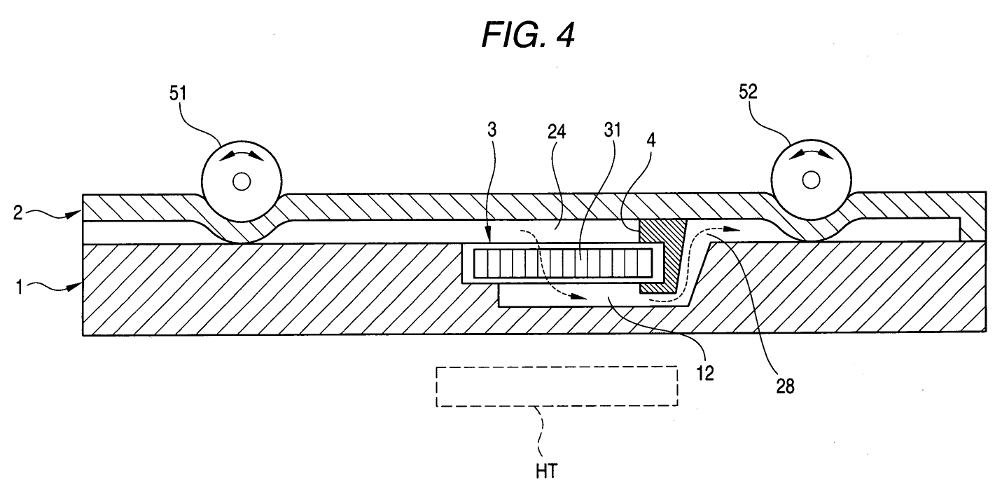

Next, operation of solution transfer in the cartridge will be described. FIG. 4 shows the operation for solution transfer.

A solution as a test target is previously injected into the chamber 21 formed in the cartridge. Injection of a solution is made by inserting an injection needle via the channel 25. The channel 25 has a plug (not shown) including an elastic body in an enclosed state. The plug is pierced with the injection needle when the solution is injected. With the solution injected, the injection needle is pulled out to stop the needle hole to secure the enclosed state.

Next, as shown in FIGS. 4 and 1, a roller 51 and a roller 52 provided in predetermined interval are used to press the elastic member 2 against the substrate 1 in order to move the roller 51 and the roller 52 in horizontal direction. With this operation, the solution injected into the chamber 21 is fed to the supply part 24 via the channel 26, the chamber 22 and the channel 27. As shown in FIG. 4, the solution penetrates the detection parts 31 of the target molecule detecting chip 3 downward from the supply part 24 and reaches the chamber 23 via the discharge part 12 and the channel 28.

Next, with the roller 51 and the roller 52 are reciprocated in a number of predetermined times while pressed against the elastic member 2 in horizontal direction in FIG. 4. With this operation, the solution is reciprocally transferred between the chamber 22 and the chamber 23 so as to cause the detection parts 31 of the target molecule detecting chip 3 being passed through a plurality times by the solution. This provides sufficient chances of the solution coming into contact with the detection part 31 required for hybridization. This also reduces hybridization errors caused by a molecule other than a target molecule. Reciprocation of the roller 51 and the roller 52 may be omitted as long as appropriate as conditions for hybridization.

As shown in FIG. 4, a heater HT may be used to keep the temperature of the solution to a value suitable for hybridization while hybridization is under way.

After the operation for hybridization is over, a predetermined read unit is used to detect target molecules that have undergone hybridization on the target molecule detecting chip 3. Target molecules may be detected inside the cartridge without the target molecule detecting chip 3 being removed therefrom. Alternatively, target molecules may be detected after the target molecule detecting chip 3 has been removed from the cartridge.

According to this embodiment, it is possible to perform hybridization by way of a target molecule detecting chip inside a cartridge. A solution is transferred to penetrate the target molecule detecting chip thus supporting a penetration type chip.

A variety of penetration type chips may be used. Examples include a chip having a probe immobilized on a porous filter (product name: Pam Gene/PAM Microarray System) from Olympus Corporation and a fiber type chip such as Genopal™ from Mitsubishi Rayon Co., Ltd.

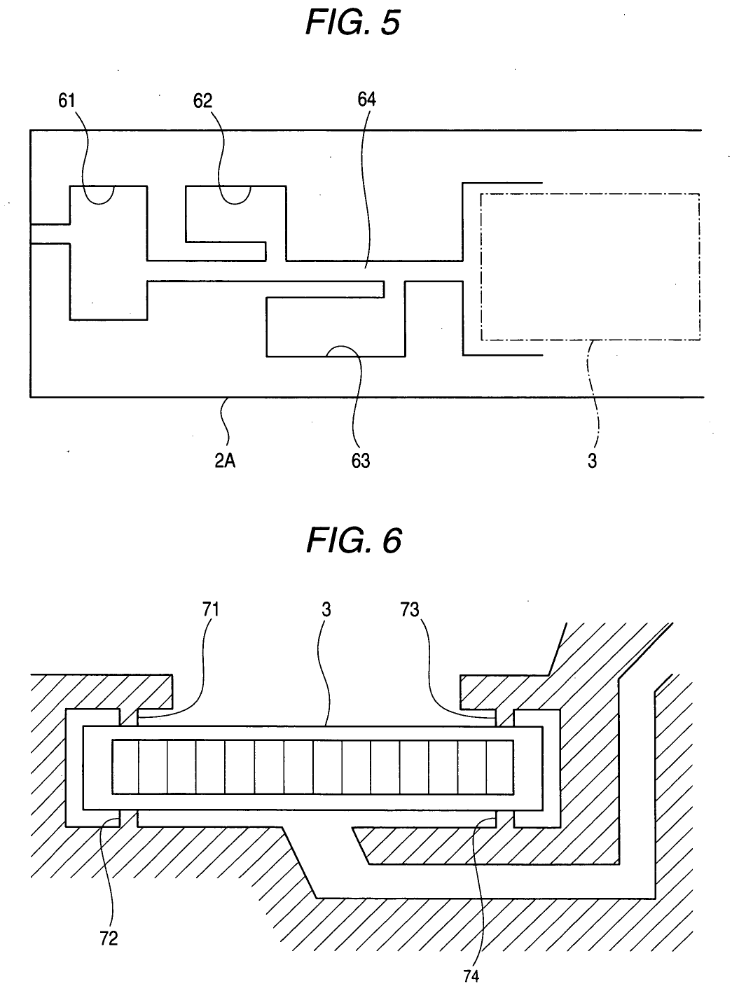

As shown in FIG. 5, there may be provided a collecting part 61 for storing a solution to be tested, a pretreatment liquid storage part 62 for previously storing a pretreatment liquid to be mixed with the solution to be tested, and a cleaning solution storage part 63 for previously storing a cleaning liquid. These parts may be connected to each other by way of the channel 64.

In this case, roller movement delivers the solution to be tested from the collecting part 61 and the pretreatment liquid from the pretreatment storage part 62 to mix them for later labeling. Next, hybridization takes place on the chip. After the hybridization, roller movement discharges the cleaning solution from the cleaning solution storage part 63 to wash non-hybridized molecules together with the solution. After that, target molecules hybridized on the chip are detected. Details of such operation are described in JP-A-2004-226068.

While the target molecule detecting chip 3 is fixed by way of bonding in this embodiment, the target molecule detecting chip 3 may be fixed by fitting to a component member of the cartridge as shown in FIG. 6. In the example of FIG. 6, the target molecule detecting chip 3 is supported via the seals parts 71 through 74 to mechanically fix the target molecule detecting chip 3 inside the cartridge while preventing leakage of the solution in the direction of thickness of the target molecule detecting chip 3 from around the target molecule detecting chip 3.

Exemplary methods for detecting target molecules hybridized on the chip include a detection method using light such as a method that is based on fluorescent measurement using laser beams and a method using a current. In case the method using a current is employed, it is possible to provide on the cartridge a connector for supplying a current into the chip inside the cartridge from the outside. In this case, the connector is attached while keeping the hermetical sealing of the cartridge.

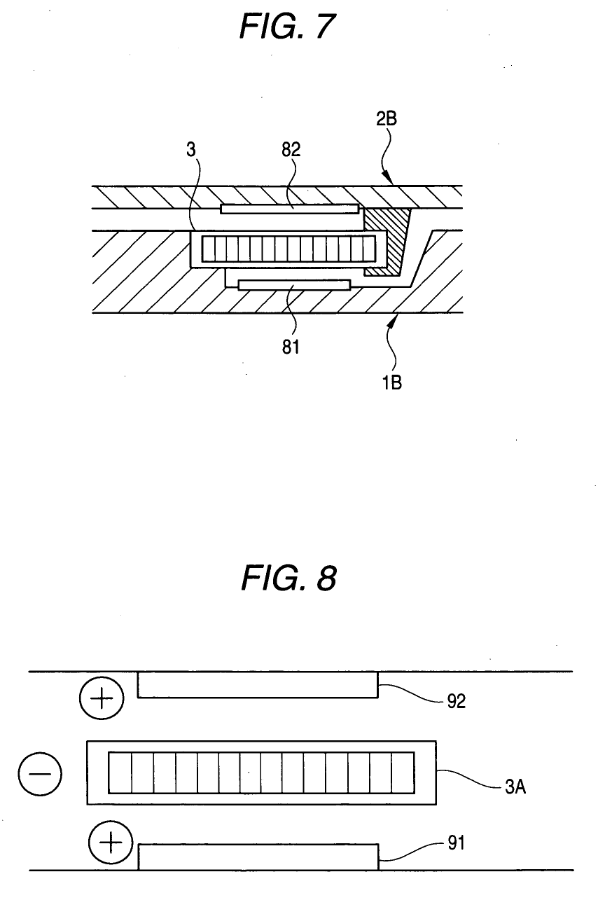

FIG. 7 shows an example where electrodes are formed on the cartridge used to apply an electric field to the chip 3 to migrate charged molecules to accelerate hybridization by the effect of electrophoresis to the charged molecules such as DNA or RNA. In the example of FIG. 7, an electrode 81 and an electrode 82 are respectively provided on the inner surface of a substrate 1B and the inner surface of an elastic member 2B. The electrode 81 and the electrode 82 are made connectable to an external power source via connectors provided on the cartridge. In this case, it is possible to invert the electric field to accelerate hybridization by repeatedly inverting a voltage applied across the electrode 81 and the electrode 82 and also reduce hybridization errors. As shown in FIG. 8, it is possible to provide a chip 3A with conductivity and use the chip 3A as an electrode. In this case, the chip 3A, the electrode 91 and the electrode 92 are connectable to an external power source via connectors provided on the cartridge.

In general, an electrode for accelerating hybridization comes in direct contact with a solution so that it is likely to be degraded thus accompanied by cumbersome work such as cleaning. This embodiment uses a cartridge disposed of each time a test is conducted so that it no longer has problems of degradation and maintenance of electrodes.

While a penetration type chip is used in the embodiment, the invention is also applicable toga plane type chip.

### Embodiment 2

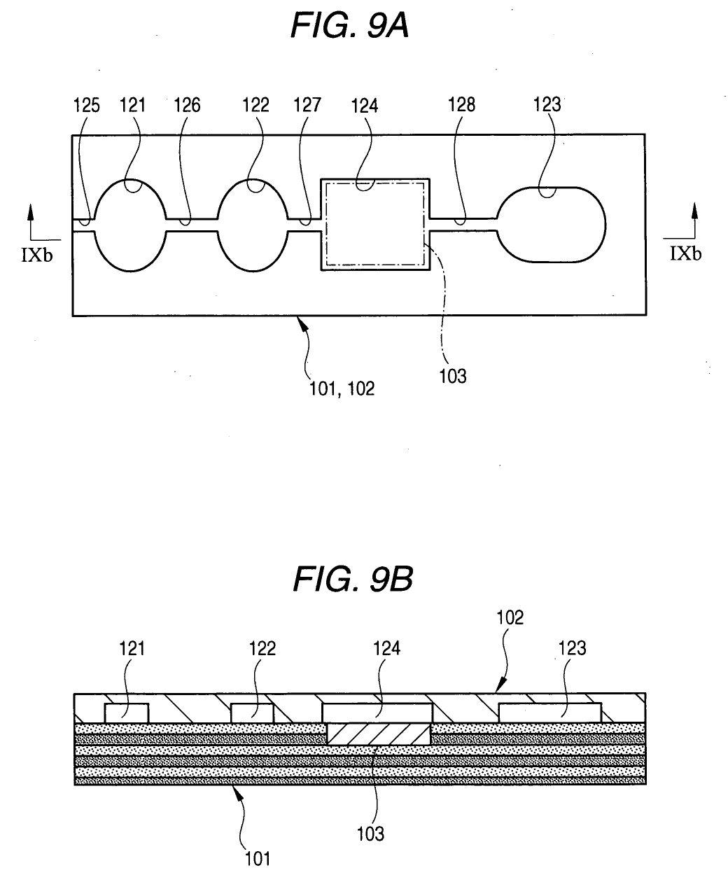

Embodiment 2 of the cartridge for chemical reaction according to the invention will be described referring to FIG. 9. This embodiment illustrates an exemplary cartridge for chemical reaction using a plane type chip.

FIG. 9A is a plan view of the cartridge for chemical reaction according to this embodiment. FIG. 9B is a cross sectional view of FIG. 9A taken along line IXb-IXb.

As shown in FIGS. 9A and 9B, the cartridge for chemical reaction according to this embodiment comprises a substrate 101 and an elastic member 102 overlaid on the substrate 101.

On the rear surface (bottom surface in FIG. 9B) of the elastic member 102 is formed a concave part of a predetermined shape recessed toward the front surface (top surface in FIG. 9B). This concave part produces space between the cartridge substrate 101 and the elastic member 102 to constitute chambers 121, 122, 123 for storing a solution, a storage part 124 for storing a chip 103 described later, and channels 125, 126, 127, 128. As shown in FIG. 9A, the channel 125, the chamber 121, the channel 126, the chamber 122, the channel 127, the storage part 124, the channel 128 and the chamber 123 serially interconnected.

In the areas other than the concave part, the cartridge substrate 101 and the elastic member 102 are bonded together. This encloses a solution stored in the concave part inside the cartridge thus preventing leakage to the outside.

As shown in FIGS. 9A and 9B, in the substrate 101 is stored a target molecule detecting chip 103.

The chip 103 is a plane type chip that detects a target molecule by way of hybridization. On the front surface (upper surface in FIG. 9B) of the chip 103 are two-dimensionally arranged detection parts to which probes corresponding to individual target molecules are fixed.

Next, operation of solution transfer in the cartridge will be described.

A solution as a test target is previously injected into the chamber 121 formed in the cartridge. Injection of a solution is made by inserting an injection needle via the channel 125. The channel 125 has a plug (not shown) including an elastic body in an enclosed state. The plug is pierced with the injection needle when the solution is injected. With the solution injected, the injection needle is pulled out to stop the needle hole to secure the enclosed state.

Next, same as Embodiment 1, rollers are moved rightward while the rollers are used to press the elastic member 102 against the substrate 101. With this operation, the solution injected into the chamber 121 is fed to the storage part 124 via the channel 126, the chamber 122, and the channel 127.

The solution fed into the storage part 124 causes hybridization on the chip 103. Same as Embodiment 1, the solution may be repeatedly supplied on the chip 103 by reciprocating the rollers.

After the operation for hybridization is over, a predetermined read unit is used to detect target molecules that have undergone hybridization on the target molecule detecting chip 103. Target molecules may be detected inside the cartridge without the target molecule detecting chip 103 being removed therefrom. Alternatively, target molecules may be detected after the target molecule detecting chip 103 has been removed from the cartridge.

According to this embodiment, it is possible to perform hybridization by way of a plane type chip inside a cartridge. In JP-A-2004-226068, detection is made with a slide glass type microarray and a cartridge housing the entire target molecule detecting chip is not used. With the invention, the entire chip is housed to provide perfect sealing. Note that the operation for hybridization disclosed in JP-A-2004-226068 may be also applied to the invention.

Same as Embodiment 1, it is possible to provide a collecting part for storing a solution to be tested and a cleaning solution storage part for previously storing a cleaning liquid may be provided.

Concerning the method for fixing the chip 103, the method for detecting target molecules and formation of electrodes on a cartridge, the structure of Embodiment 1 may be applied to this embodiment.

In the foregoing embodiments, types of a target molecule are not limited. For example, target molecules may be DNA, RNA, protein and metabolites. The invention is not limited to detection of biopolymers but is applicable to general chromatography.

The scope of the invention is not limited to the above embodiments. The invention is widely applicable to a cartridge for chemical reaction that causes chemical reaction by way of deformation caused by external forces to deliver an internal solution.

What is claimed is:

1. A cartridge for chemical reaction that causes chemical reaction by feeding an internal liquid by way of deformation caused by external forces, comprising:
a target molecule detecting chip which is entirely housed in the cartridge for chemical reaction, and
channels for feeding the internal liquid, being connected to the target molecule detecting chip.

2. The cartridge for chemical reaction according to claim 1, wherein
the target molecule detecting chip is a penetration type chip that allows a solution including a target molecule to penetrate the chip.

3. The cartridge for chemical-reaction according to claim 2, wherein
the target molecule detecting chip allows the solution to pass through the inside of the penetration type chip by way of deformation caused by external forces applied to the cartridge for chemical reaction.

4. A cartridge for chemical reaction that causes chemical reaction by feeding an internal liquid by way of deformation caused by external forces,
the cartridge for chemical reaction comprising:
a penetration type target molecule detecting chip that allows a solution including a target molecule to penetrate the chip, and
channels for feeding the internal liquid, being connected to the target molecule detecting chip, wherein
the target molecule detecting chip allows the solution to pass through the inside of the target molecule detecting chip by way of deformation caused by external forces applied to the cartridge for chemical reaction.

5. The cartridge for chemical reaction according to claim 1, wherein
the target molecule detecting chip detects any one of DNA, RNA, protein and metabolite as a target molecule.

6. The cartridge for chemical reaction according to claim 1, wherein
the cartridge for chemical reaction includes electrodes for applying an electric field to charged molecules inside the cartridge for chemical reaction.

7. The cartridge for chemical reaction according to claim 4, wherein
the target molecule detecting chip detects any one of DNA, RNA, protein and metabolite as a target molecule.

8. The cartridge for chemical reaction according to claim 4, wherein
the cartridge for chemical reaction includes electrodes for applying an electric field to charged molecules inside the cartridge for chemical reaction.
